(19) United States
(12) Patent Application Publication
(10) Pub. No.: US 2019/0359922 A1
(43) Pub. Date: Nov. 28, 2019

(54) ETHANOL PRODUCTION, USE, AND WASTE RECOVERY USING AQUATIC PLANTS

(71) Applicant: BCS, LLC, Sioux Falls, SD (US)

(72) Inventors: Tony A. Hagen, Sioux Falls, SD (US); Cecil Massie, Bloomington, MN (US); Nicolas Hagen, Sioux Falls, SD (US)

(73) Assignee: BCS, LLC, Sioux Falls, SD (US)

(21) Appl. No.: 16/290,632

(22) Filed: Mar. 1, 2019

Related U.S. Application Data
(63) Continuation of application No. 14/629,340, filed on Feb. 23, 2015, now abandoned.
(60) Provisional application No. 61/943,943, filed on Feb. 24, 2014.

(51) Int. Cl. C12M 1/00; C12M 1/12

(52) U.S. Cl. CPC C12M 21/12; C12M 21/02; C12M 25/00; C12M 29/00

(57) ABSTRACT

Embodiments of the present disclosure relate to systems and methods for ethanol production, use, and waste recovery using aquatic plants. In certain embodiments, systems and methods are disclosed for reuse and further processing of waste alcohols, sugars, organic acids and/or byproducts produced by conversion of corn, other grains, or other biomass materials of use in biofuel production methods.

### CROSS REFERENCE TO RELATED APPLICATIONS

This application is a continuation application of U.S. application Ser. No. 14/629,340, filed Feb. 23, 2015, which claims the benefit under 35 USC § 119(e) to U.S. Provisional Application Ser. No. 61/943,943 filed on Feb. 24, 2014, each of which is incorporated herein by reference in their entirety.

### FIELD OF INVENTION

Embodiments of the present disclosure relate to systems and methods for ethanol production, use, and waste recovery using aquatic plants. In certain embodiments, systems and methods are disclosed for reuse and further processing of waste alcohols, sugars, organic acids and/or byproducts produced by conversion of corn, other grains, or other biomass materials of use in biofuel production methods.

### BACKGROUND

Current ethanol production processes rely primarily on the direct conversion of biomass sources into ethanol. In grain-based ethanol production, for example, a grain such as corn is mechanically, thermally and/or chemically processed, and a fraction extracted from the processed grain is placed in fermentation tanks containing microbes. The fermented extract is then distilled in order to produce biofuels and/or processed biomass for other uses.

Certain drawbacks to these conventional ethanol or alcohol production methods can include high raw material (e.g., grain) consumption, large by-product production, and consumption of excess water and energy. Accordingly, alternatives to convention ethanol or alcohol production methods have been sought.

### SUMMARY

The present disclosure provides systems and methods for producing ethanol, using organic compounds including ethanol, and recovering useful products from waste of these processes using aquatic plants. In certain embodiments, methods disclosed herein are used to produce a high grade or pure grade ethanol of use in a variety of applications. In other embodiments, waste produced by generating ethanol from crops, for example, cellulosic biomass crops such as corn crops produces biomass or waste alcohols (e.g. ethanol) are harvested and further used to feed and/or be further processed to reduce waste production by such crops.

Other crops known to produce ethanol include, but are not limited to, a variety of feedstocks such as sugar cane, bagasse, miscanthus, sugar beet, sorghum, grain, switchgrass, barley, hemp, kenaf, potatoes, sweet potatoes, cassava, sunflower, fruit, molasses, corn, stover, grain, wheat, straw, cotton, other biomass, as well as many types of cellulose waste and harvesting.

According to an exemplary embodiment of the present disclosure, a system is provided for producing ethanol. The system includes a container, a substrate in the container, at least one water inlet to the container to deliver water to the container, at least one carbon dioxide inlet to the container, the at least one carbon dioxide inlet communicating with an adjacent facility (e.g., an industrial plant or combustion facility) to receive carbon dioxide from the facility, at least one aquatic plant having a root portion anchored into the substrate, the at least one aquatic plant performing a metabolic photosynthesis process to convert the carbon dioxide from the facility into glucose and a metabolic fermentation process to convert the glucose into ethanol, and at least one outlet from the container to remove the water and the ethanol from the container.

According to another exemplary embodiment of the present disclosure, another system is provided for producing ethanol. The system includes a container, a substrate in the container, at least one water inlet to the container to deliver water to the container, at least one carbon inlet to the container, the at least one carbon inlet communicating with an adjacent facility to receive a waste carbon material from the adjacent facility, at least one aquatic plant having a root portion anchored into the substrate, the at least one aquatic plant converting the waste carbon material into glucose and converting the glucose into ethanol, and at least one outlet from the container to remove the water and the ethanol from the container.

According to yet another exemplary embodiment of the present disclosure, an additional system is provided for producing ethanol. The system includes a container, a substrate in the container, the substrate comprising at least one elongate plastic material arranged irregularly in the container to define a plurality of spaces, at least one aquatic plant anchored into the plurality of spaces in the substrate, at least one water inlet to the container to deliver water to the container, and at least one water outlet from the container to remove water and ethanol from the container.

In other embodiments, waste carbon dioxide of use in the above referenced systems can be derived from landfills, wastewater facilities, cement facilities, or syngas from coal production or a naturally produced carbon dioxide.

### BRIEF DESCRIPTION OF THE DRAWINGS

The above-mentioned and other features and advantages of this disclosure, and the manner of attaining them, will become more apparent and the invention itself will be better understood by reference to, but not limited by, the following description of embodiments of the invention taken in conjunction with the accompanying drawings, wherein:

FIG. 1 is a schematic view of a system for cultivating and producing ethanol from aquatic plants;

FIG. 2 is a schematic view of the system of FIG. 1 in communication with an adjacent combustion facility;

FIG. 3 is a schematic view of the system of FIG. 1 in communication with an adjacent first-generation ethanol facility; and

FIGS. 4A and 4B show exemplary substrates for use in certain embodiments disclosed herein as well as in the system of FIG. 1.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

### System Overview

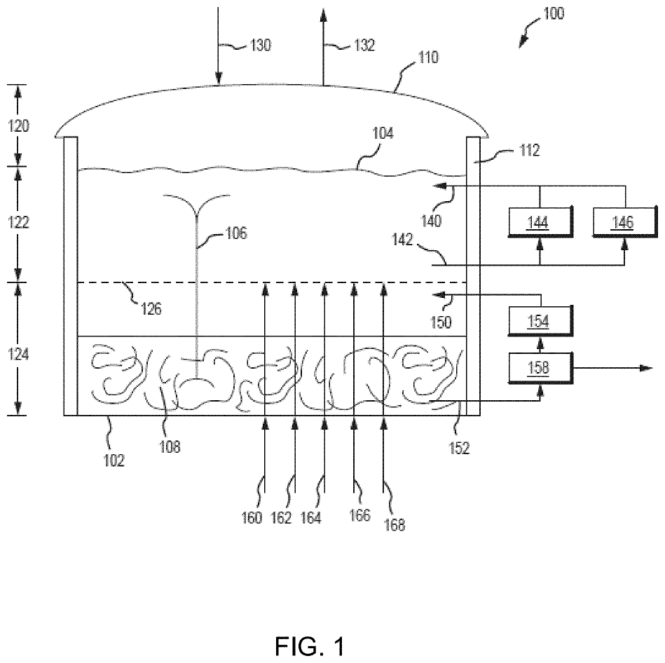

FIG. 1 provides a system 100 for cultivating and producing ethanol from aquatic plants 106. System 100 includes one or more containers 102. Each container 102 holds water or a water composition 104, one or more aquatic plants 106, and a substrate 108 configured to anchor and support root growth of the aquatic plants 106.

The shape, dimensions, and configuration of each container 102 may vary depending on the number and type(s) of aquatic plants 106 being used in system 100 and the surrounding environment. For example, the depth of container 102 may be chosen to promote growth of the particular type(s) of aquatic plants 106 being used in system 100. It is to be understood that container 102 may be scaled to accommodate available materials, equipment, and space. In some cases, the depth of each container 102 can range from about 10 cm to about 20 m (e.g., 10 cm to 100 cm, 50 cm to 1 m, 100 cm to 1 m, 500 cm to 3 m, 1 m to 5 m, 4 m to 10 m, 5 m to 7 m, 5 m to 10 m, or 10 m to 20 m).

Container 102 may be constructed or lined with any suitable water-tight material to prevent leakage of fluids and gases from container 102. Suitable materials include, without limitation, concrete, plastic, rubber, metal, glass, fiberglass, earth-filled berm, or the like.

The type of water 104 used in container 102 may vary. For example, water or a water composition 104 may include fresh water, salt water, wastewater and brackish water. In certain embodiments, water 104 may be wastewater that is obtained from another facility, such as a food processing facility. The water 104 may include dissolved gases and nutrients.

The aquatic plants 106 used in container 102 may be selected from any number of aquatic plants which readily live in or on aquatic environments, such as directly in water or in permanently saturated soil, and that excrete ethanol under the conditions described herein. Exemplary aquatic plants 106 include submersed aquatic herbs from the Potamogeton family (e.g., Potamogeton pectinatus, also known as Stuckenia pectinata or Sago pondweed), the Aponogeton family, or the like. For example, any submerged aquatic plant capable of using waste from a biofuel producing facility such as biomass waste or ethanol gas for further use and/or processing is contemplated herein.

Depending on various factors, including (but not limited to) the type of environment and/or type of water used with system 100, other suitable aquatic plants may include, for example, algae, submersed aquatic herbs such as, but not limited to, Stuckenia vaginata, Stuckenia filiformis, Potamogeton crispus, Potamogeton distintcus, Potamogeton nodosus, Ruppia maitima, Myriophyllum spicatum, Hydrilla verticillata, Elodea densa, Hippuris vulgaris, Aponogeton boivinianus, Aponogeton rigidifolius, Aponogeton longiplumulosus, Didiplis diandra, Vesicularia dubyana, Hygrophilia augustifolia, Micranthemum umbrosum, Eichhornia azurea, Saururus cernuus, Cryptocoryne lingua, Hydrotriche hottoniiflora, Eustralis stellata, Vallisneria rubra, Hygrophila salicifolia, Cyperus helferi, Cryptocoryne petchii, Vallisneria americana, Vallisneria torta, Hydrotriche hottoniiflora, Crassula helmsii, Limnophila sessiliflora, Potamogeton perfoliatus, Rotala wallichii, Cryptocoryne becketii, Blyxa aubertii and Hygrophila difformmis, duckweeds such as, but not limited to, Spirodela polyrrhiza, Wolffia globosa, Lemna trisulca, Lemna gibba, Lemna minor, and Landoltia punctata, water cabbage, such as but not limited to Pistia stratiotes, buttercups such as but not limited to Ranunculus, water caltrop such as but not limited to Trapa natans and Trapa bicornis, water lily such as Nymphaea lotus, Nymphaeaceae and Nelumbonaceae, water hyacinth such as but not limited to Eichhornia crassipes, Bolbitis heudelotii, and Cabomba, and seagrasses such as but not limited to Heteranthera zosterifolia, Posidoniaceae, Zosteraceae, Hydrocharitaceae, Cymodoceaceae, and hybrids of such plants. Moreover, in one of the various embodiments, a host algae may be selected from the group consisting of green algae, red algae, brown algae, diatoms, marine algae, freshwater algae, unicellular algae, multicellular algae, seaweeds, cold-tolerant algal strains, heat-tolerant algal strains, ethanol-tolerant algal strains, and combinations thereof.

The aquatic plants 106 may be obtained and placed in container 102 in any conventional manner. For example, the aquatic plants 106 may be gathered from lakes or ponds, grown in holding tanks, or grown directly in container 102. In some embodiments, the aquatic plants 106 are non-genetically modified plants. In other embodiments, the aquatic plants 106 are genetically modified plants. Genetic modifications may include, without limitation, the inclusion of a transgene or up-regulation or down-regulation of a target gene that confers resistance to a pest, resistance to a pesticide or herbicide, tolerance to heat, tolerance to cold, improved biofuel processing (e.g. ethanol production), improved biofuel production, and/or tolerance to high concentrations of plant byproducts (e.g., ethanol or other biofuel).

Container 102 may include a cover 110, as shown in FIG. 1. Cover 110 may serve as a light barrier that controls the passage of photosynthesis-inducing light to the aquatic plants 106 in container 102. In certain embodiments, cover 110 may be selectively applied to container 102 to block the passage of photosynthesis-inducing light into container 102 and removed from container 102 to allow the passage of photosynthesis-inducing light into container 102. In other embodiments, cover 110 may be permanently applied to container 102 to block the passage of natural photosynthesis-inducing light (e.g., sunlight) into container 102. In this embodiment, an artificial light source (not shown) may be provided beneath cover 110 to selectively provide photosynthesis-inducing light to container 102. It is to be understood based on the present disclosure that the cover 110 can, but is not required to, inhibit all light from reaching a plant of the system. Rather the cover 110 may only inhibit light at a wavelength or intensity that would induce photosynthesis in a plant of the system. For example, the cover 110 can be a filter that allows only wavelengths that do not induce photosynthesis to pass. Examples of wavelengths that induce photosynthesis include wavelengths from about 380 nm to about 710 nm. Depending on the plant being used, the range of wavelengths that induce photosynthesis can be broader or narrower, but can be ascertained using known methods. In one embodiment, a sealing barrier and the cover 110 can constitute a single structure that may or may not be separable.

Cover 110 may also serve as an air barrier that controls the passage of air, specifically oxygen and carbon dioxide, from the surrounding atmosphere to the aquatic plants 106 in container 102. In other embodiments, a separate air barrier (not shown) may be provided.

The passage of photosynthesis-inducing light to the aquatic plants 106 in container 102 may also be controlled by adding a light-reducing dye to water 104. The dye may allow surface light to travel through an upper or shallower section of water 104 (e.g., about 2-6 inches from the surface), but may prevent the light from traveling through a lower or deeper section of water 104 (e.g., more than about 6 inches from the surface). Such dyes may prevent or reduce algae growth and growth of other undesired materials in container 102.

The temperature of water 104 in container 102 may be controlled. For example, water 104 in container 102 may be maintained at a temperature of about 50° F. to about 85° F. In FIG. 1, container 102 is at least partially surrounded by an insulating material 112. In embodiments where container 102 is partially or fully buried underground, the insulating material 112 may include the surrounding soil. Other suitable insulating materials 112 include foam, fiberglass, puncture-resistant geotextile fiber or mat liners, water, super-absorbent polymer beads, an air-filled tube, and the like. In certain embodiments, container 102 may be unevenly insulated. For example, a lower section of the insulating material 112 may have a different thermal resistance (R-value) than an upper section of the insulating material 112.

Container 102 may include a plurality of zones or regions. In the illustrated embodiment of FIG. 1, container 102 includes a first region 120 including a predominantly gaseous headspace above water 104, a second region 122 including an upper section of water 104 that alternates between an aerobic (i.e., oxygenated) condition and an anaerobic (i.e., oxygen depleted) condition, and a third region 124 including a lower section of water 104 that generally maintains an anaerobic condition. Root portions (e.g., tubers, rhizomes) of the aquatic plants 106 and substrate 108 may be located predominantly or entirely in the third region 124 of container 102. Stem and leaf portions of the aquatic plants 106 may extend upwardly into the second region 122 of container 102. The substrate 108 can be embossed, sculpted, or carved for increased surface area.

Container 102 may include various forms of bacteria and fungus to facilitate metabolic processes of the aquatic plants 106. The bacteria and fungus may be aerobic or anaerobic, depending on the condition of the surrounding water 104. For example, because substrate 108 is located in the anaerobic third region 124 of container 102 in FIG. 1, substrate 108 may be inoculated with anaerobic bacteria and fungus.

The physical and chemical characteristics of the first region 120, second region 122, and third region 124 of container 102 may be controlled independently and distinctly. For example, the composition, temperature, pH, oxidation/reduction potential (ORP), ion concentration, conductivity, bacteria content, dissolved mineral content, and/or dissolved gas content of each of the first region 120, second region 122, and third region 124 of container 102 may be controlled independently and distinctly, for example to optimize conditions for the plants.

A boundary 126 may be provided between the second region 122 and the third region 124 to prevent mixing of water 104 and to maintain different conditions between the second region 122 and the third region 124. In certain embodiments, boundary 126 may be achieved by providing water 104 in the second region 122 at a lower density than the water 104 in the third region 126, such as by varying the dissolved mineral content, salinity, and/or the temperature of water 104. For example, a temperature difference of about 4-6 degrees Fahrenheit between water 104 in the second region 122 and the third region 124 may be sufficient to maintain boundary 126 between the two regions. In other embodiments, boundary 126 may be achieved by providing a physical barrier between the second region 122 and the third region 124. The barrier may be porous to allow aquatic plants 106 to grow therethrough or may be a solid material capable of removal. Suitable barriers may include viscous liquids (e.g., gelatins, waxes, carbohydrate solutions), woven or non-woven fabrics, paper, plastic or nylon screens, plastic matrices, and the like.

As discussed further below, system 100 may include various inlets and outlets in communication with regions 120, 122, 124 of container 102 to control the flow of materials to and from container 102. In addition to the inlets and outlets shown in FIG. 1, system 100 may also include pumps, flow control valves, heat exchangers, filters, storage units, and other equipment to facilitate the flow of materials to and from container 102 and/or allow for easy collection.

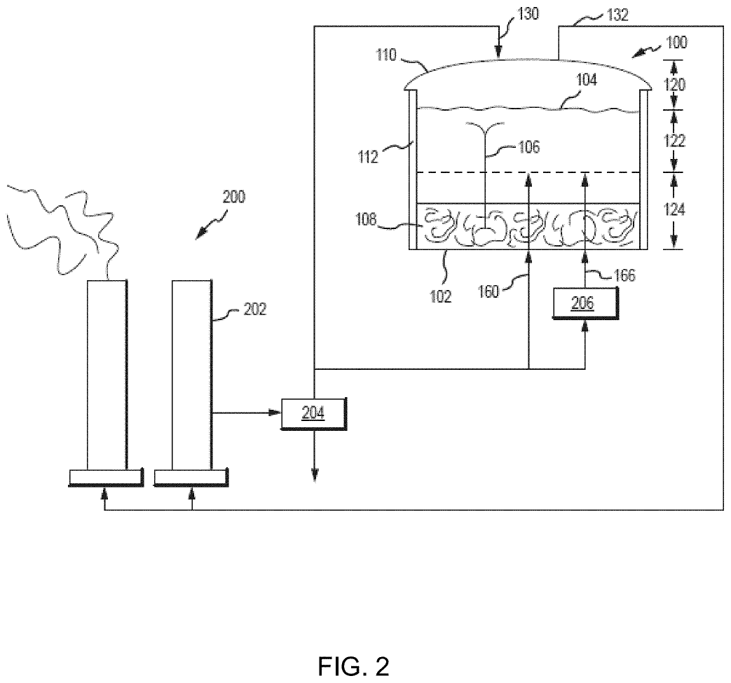

First region 120 of container 102 illustratively includes a carbon dioxide gas inlet 130 and an oxygen gas outlet 132. The oxygen-rich gas that is removed from the oxygen gas outlet 132 may be purified, stored, and/or distributed for use, such as to combustion systems and/or fishery systems. According to an exemplary embodiment of the present disclosure, and as discussed further below with respect to FIG. 2, the oxygen-rich gas from oxygen gas outlet 132 may be supplied to a combustion facility 200, and carbon dioxide flue gas from the combustion facility 200 may be returned to system 100.

Second region 122 of container 102 illustratively includes a water inlet 140 and a water outlet 142 to circulate water through container 102. The water inlet 140 is in selective communication with an aerobic water source 144 and an anaerobic water source 146 to supply aerobic water or anaerobic water to second region 122 of container 102, as desired. The water outlet 142 is also in selective communication with the aerobic water source 144 and the anaerobic water source 146 to remove water from second region 122 of container 102 and to return the water to the appropriate aerobic water source 144 or anaerobic water source 146. Before reintroducing any water back to container 102, the water may be treated using ultraviolet light, antibiotics, and/or algaecides, for example. Also, the water may be processed to add or remove oxygen, as desired. For example, oxygen may be injected into the water returning to the aerobic water source 144, and oxygen may be removed from the water returning to the anaerobic water source 146. Further, the water may be pre-filtered through a filtration device.

Third region 124 of container 102 illustratively includes a water inlet 150 and an ethanol-water outlet 152. The water inlet 150 is in communication with an anaerobic water source 154 to supply anaerobic water to third region 124 of container 102. In certain embodiments, the anaerobic water source 154 to third region 124 may be the same as the anaerobic water source 146 to second region 122. It is also within the scope of the present disclosure that the water inlet 150 to third region 124 may communicate in an alternating fashion with an aerobic water source (not shown), like the water inlet 140 to second region 122. The ethanol-water outlet 152 is in communication with an ethanol separation apparatus 158 (FIG. 1) to separate ethanol from water using distillation, condensation, filtration, absorption, or other separation techniques. The separated water may be directed to the anaerobic water source 154 and returned to third region 124 of container 102. The separated ethanol may be purified, stored, and/or distributed for use.

Second region 122 and/or third region 124 of container 102 may also include one or more of the following carbon inlets to supply desirable levels and forms of carbon to water 104 for use by the aquatic plants 106: a carbon dioxide gas inlet 160, an aqueous carbon dioxide inlet 162, a carbonic acid inlet 164, a bicarbonate or carbonate inlet 166, and an organic or inorganic carbon matter inlet 168 (FIG. 1). It is also understood that carbon dioxide gas may enter water 104 from the gaseous headspace or first region 120 of container 102.

The carbon inlets 160, 162, 164, 166, 168 may be configured to introduce carbon to container 102 as a gas, a liquid solution, or a solid powder, as appropriate. The carbon dioxide gas inlet 160, for example, may be configured to inject the carbon dioxide gas as bubbles into water 104 in container 102.

Carbon dioxide has limited solubility in water 104, so using other carbon inlets (such as the carbonic acid inlet 164, the bicarbonate or carbonate inlet 166, and/or the organic or inorganic carbon matter inlet 168) may make more carbon available in water 104 for use by the aquatic plants 106. According to an exemplary embodiment of the present disclosure, the carbonate concentration in water 104 may be maintained at or above about 5 millimols per liter (mmol/L), about 10 mmol/L, or about 15 mmol/L.

Additional information regarding system 100 is disclosed in U.S. Patent Application Publication No. 2013/0071902 to Hagen, the disclosure of which is expressly incorporated herein by reference in its entirety.

### Metabolic Processes

Once established in container 102, aquatic plants 106 undergo various metabolic processes, including photosynthesis, respiration, and fermentation. Each of these metabolic processes is discussed further below.

During photosynthesis, the aquatic plants 106 consume carbon dioxide (CO₂) and produce oxygen (O₂) and carbohydrates, specifically glucose (C₆H₁₂O₆), as shown in Reaction (1) below. Photosynthesis generally takes place in the presence of light and oxygen and is an aerobic metabolic process. Photosynthesis is the energy collection and storage process for the aquatic plants 106.

6CO₂+6H₂O→6CO₂+C₆H₁₂O₆ (1)

Photosynthesis may be facilitated in system 100 by allowing photosynthesis-inducing light to reach the aquatic plants 106 and/or by providing aerobic (i.e., oxygenated) water 104 to the aquatic plants 106. In FIG. 1, for example, cover 110 may be removed from container 102 to expose the aquatic plants 106 to natural or artificial light, and aerobic water may be directed to at least second region 122 of container 102 from the aerobic water source 144. Additional information regarding methods and systems to facilitate photosynthesis is disclosed in U.S. Patent Application Publication No. 2011/0086400 to Hagen, the disclosure of which is expressly incorporated herein by reference in its entirety.

During respiration, the aquatic plants 106 consume oxygen (O₂) and the glucose (C₆H₁₂O₆) from photosynthesis and produce carbon dioxide (CO₂), as shown in Reaction (2) below.

6O₂+C₆H₁₂O₆→6CO₂+6H₂O (2)

Respiration is the opposite of photosynthesis. In nature, photosynthesis generally occurs during daytime hours with the aquatic plants 106 deriving energy from sunlight or another light source, and respiration generally occurs during the nighttime hours with the aquatic plants 106 deriving energy from stored carbohydrates. Depending on the time spent undergoing respiration, the particular type(s) of aquatic plants 106 being used in system 100, and other factors, the aquatic plants 106 may consume about 40%, 50%, or 60% of the glucose generated from photosynthesis during respiration over the course of a day. In some cases, light can be added at an intensity that is at or above the light compensation point (LCP), which may prevent any significant increase in the rate of photosynthesis.

During fermentation, the aquatic plants 106 metabolize the stored glucose (C₆H₁₂O₆) into ethanol (C₂H₅OH), as shown in Reaction (3) below. In some cases, decreasing the pH can cause a shift from acetate-ethanol fermentation to lactate-ethanol fermentation. The aquatic plants 106 may also elongate during fermentation to form cellular chambers for storage of additional carbohydrates created during subsequent photosynthesis. Fermentation generally takes place in a dark and anaerobic environment and is an anaerobic metabolic process.

C₆H₁₂O₆→2CO₂+2C₂H₅OH (3)

As used herein, an “anaerobic” environment has a level of oxygen depletion that induces the aquatic plants 106 to enter or maintain the anaerobic metabolic fermentation process. Thus, an “anaerobic” environment may be sufficient to reduce or maintain a level of intracellular oxygen in the aquatic plants 106 to facilitate an anaerobic metabolic fermentation process. It should be understood that the term “anaerobic” does not necessarily indicate a complete absence of oxygen in the water 104, as a very small quantity of oxygen will likely be dissolved in the water 104.

Fermentation may be facilitated in system 100 by inhibiting photosynthesis-inducing light from reaching the aquatic plants 106 and/or by providing anaerobic (i.e., oxygen depleted) water 104 to the aquatic plants 106. In FIG. 1, for example, cover 110 may be applied to container 102 to block or inhibit unwanted photosynthesis-inducing light from reaching the aquatic plants 106, and anaerobic water may be directed to second region 122 of container 102 from the anaerobic water source 146. Anaerobic water may also be directed to third region 124 of container 102 from the anaerobic water source 154. In addition to inhibiting photosynthesis-inducing light from entering container 102, cover 110 may also inhibit unwanted oxygen from the surrounding air from entering the water 104 in container 102. Depriving container 102 of light may suppress or prevent photosynthesis, and depriving container 102 of oxygen may suppress or prevent respiration.

Alternatively or additionally, oxygen reducing additives such as corn, yeast, bacteria (e.g., genetically altered bacteria and/or bacteria capable of fermentation), or enzymes, which consume oxygen and sugars while producing carbon dioxide, may be added to the cell to deplete the oxygen levels. In order to promote the depletion of oxygen levels, a secondary carbohydrate source, for instance corn, molasses, wheat or other sources of sugar, may be added to the water for use by the oxygen reducing additives. The secondary carbohydrate source may be added along with yeast to cause a strong enough reaction to remove a significant amount of oxygen from the system. One benefit of the reduction of oxygen may be additional production of ethanol by the oxygen reducing additives.

The lack of sufficient oxygen in the water facilitates the anaerobic process in the aquatic plants causing them to metabolize carbohydrates and to produce ethanol. The production of ethanol may be further encouraged by the introduction of chemical catalysts and CO₂. Suitable chemical catalysts include acetic acid and 2,4-dichlorophenoxyacetic acid (known generically as 2,4-D). CO2 may be obtained from waste sources such as electricity facilities and petroleum refineries. Additional nutrients and salts such as salts of potassium, nitrogen and phosphorus may further be added to promote growth of the aquatic plants. Further, depending upon the species of aquatic plant being utilized, organic substrates, including but not limited to those such as sucrose, glucose and acetate, may also be added to the cell. Additional information regarding methods and systems to facilitate fermentation is disclosed in the above-incorporated U.S. Patent Application Publication No. 2011/0086400.

According to one embodiment of the present disclosure, system 100 may encourage the aquatic plants 106 in container 102 to alternate repeatedly between photosynthesis and fermentation over time, as disclosed in the above-incorporated U.S. Patent Application Publication No. 2011/0086400. For example, after several days or weeks of facilitating fermentation in container 102, the condition of system 100 may alternate to begin facilitating photosynthesis in container 102. Then, after several days or weeks of facilitating photosynthesis in container 102, the condition of system 100 may alternate once again to return to facilitating fermentation in container 102. As discussed above, facilitating photosynthesis may involve removing cover 110 from container 102 and/or directing aerobic water into second region 122 of container 102, and facilitating fermentation may involve removing cover 110 from container and/or directing anaerobic water into second region 122 of container 102. This alternating process creates a self-sustaining cycle as the plant growth replenishes both plant matter lost to plant senescence and those plants which no longer meet established tolerances of ethanol production.

Various means for regulating (e.g., selectively blocking/allowing) photosynthesis inducing light to reach the aquatic plant may be utilized. Such means include, for instance, barriers, covers, domes or other enclosure structure, which serve as a light barrier at least during the anaerobic process. These aforementioned barriers, covers, etc., may be removable when it is no longer desired to maintain the aquatic plant in an anaerobic condition. In one embodiment, the cells are illuminated by light visible to humans but which facilitates the “dark” condition for the plant. Other suitable regulation means include light filters that diffuse photosynthesis inducing light. Artificial lights sources may be used to preserve the dark condition and/or to selectively allow photosynthesis when the anaerobic condition is not desired. In some embodiments, a gradual transition from “light” conditions to “dark” conditions and/or vice-versa is desirable to reduce the risk of shocking the plant.

Optionally, in conjunction with the dark phase, the oxygen content of the cell can be reduced by introducing water into the cell that is severely depleted (i.e. rendered anoxic) of oxygen through the use of organic, chemical, or mechanical means. This may also be accomplished by removing oxygen from water contained in the cell. It should be understood that the term “anoxic” does not necessarily indicate a complete absence of oxygen in the water, as a very small quantity of oxygen will likely be dissolved in the water.

In other embodiments, the anaerobic process described above is preceded by, followed by or alternated with an aerobic process. The aerobic process is initiated and/or facilitated in the aquatic plant by creating an oxygenated condition in the cell, which facilitates the production and storage of carbohydrates by the aquatic plant. This oxygenated condition may be created by a variety of approaches, which may be used independently or in combination. In one embodiment, oxygenated water is added to the cell or oxygen is directly introduced into water contained in the cell. In another embodiment, the gas barrier is removed to allow the oxygen concentration of the water to naturally increase. Accordingly, the oxygenated condition may be accomplished by introducing oxygenated water into the cell, by removing anoxic water and/or allowing the water to oxygenate naturally by plant releasing of oxygen and exposure to an oxygenated atmosphere.

Embodiments of the invention can be practiced with multiple cells wherein anoxic water and oxygenated water are rotated between the cells as needed to alternate between an anoxic condition and an oxygenated condition. For example, the process of utilizing multiple cells may include a first cell having anoxic water containing 2% ethanol, which is moved into a second cell having previously been oxygenated. The anoxic water replaces the removed oxygenated water in the second cell to create an anoxic condition in the second cell. Within the second cell plant growth and ethanol production are then stimulated. It is noted based on the present disclosure that having ethanol originally in the second cell (since the anoxic water contains ethanol from the anaerobic process of the first cell) may further spur ethanol production when the aquatic plants detect ethanol in the water. The ethanol concentration may be allowed to increase, for example, up to 4% in the second cell. Each time the anoxic water is moved into a new cell, the elongation and ethanol production of those plants is stimulated. Once the ethanol concentration of the anoxic water reaches a predetermined level, such as for example 10% by volume, the anoxic water is removed from the cell and the ethanol extracted from the water using conventional means.

### Carbon Sources for Photosynthesis

During photosynthesis, the aquatic plants 106 consume carbon dioxide, as discussed above with respect to Reaction (1). As shown in FIG. 2, the carbon dioxide that is supplied to container 102 for photosynthesis may be obtained from an adjacent combustion facility 200, such as an electrical facility or a petroleum refinery. More specifically, the carbon dioxide may be obtained from an exhaust or flue 202 of the combustion facility 200. In certain embodiments, the combustion facility 200 may also be fueled by the oxygen-rich gas from oxygen gas outlet 132 of container 102, resulting in material being transferred both to and from the combustion facility 200.

As shown in FIG. 1, a separator 204 may be provided downstream of flue 202 to remove water vapor and other components from the flue gas. The separator 204 may be a condenser, for example, that cools and condenses the water vapor to provide a dewatered carbon dioxide product.

The carbon dioxide from the flue 202 may be introduced to container 102 in different forms. In one embodiment, the carbon dioxide may be introduced to container 102 in the form of a gas. In this embodiment, the carbon dioxide may be introduced to first region 120 of container 102 via the carbon dioxide gas inlet 130 or to second region 122 and/or third region 124 of container 102 via the carbon dioxide gas inlet 160. In another embodiment, the carbon dioxide may be converted to a bicarbonate or carbonate material upstream of container 102, such as by directing the carbon dioxide through a reactor 206 containing potassium hydroxide, sodium hydroxide, or suitable enzymes. In this embodiment, the bicarbonate or carbonate material, which may be in the form of a liquid solution or a solid powder, for example, may be introduced to second region 122 and/or third region 124 of container 102 via the bicarbonate or carbonate inlet 166.

Advantageously, the combustion facility 200 may supply the flue gas to container 102 without purifying the flue gas or releasing the flue gas into the atmosphere. Also, the combustion facility 200 may supply the flue gas to container 102 without having to heat the flue gas sufficiently to rise from flue 202, which may save the combustion facility 200 up to 5%, 10%, or more in energy savings.

### Carbon Sources for Fermentation

During fermentation, the aquatic plants 106 metabolize glucose into ethanol, as discussed above with respect to Reaction (3). The aquatic plants 106 may be heterotrophs that are capable of taking in and converting other carbon-containing materials to glucose, and then ultimately to ethanol. Such carbon-containing materials may be supplied to the aquatic plants 106 via the organic or inorganic carbon matter inlet 168 of FIG. 1. The root portions of the aquatic plants 106 may especially well-suited to take in these other carbon-containing materials, so the organic or inorganic carbon matter inlet 168 may direct the materials into third region 124 of container 102 to interact with the root portions of the aquatic plants 106, in particular. Stress hormones (e.g., IAA, ABA, GA) may be used to encourage heterotrophic consumption of the carbon-containing materials.

Suitable carbon-containing materials include, for example, carbohydrates (e.g., starch), sugars (e.g., glucose, sucrose, and fructose), aldehydes, alcohols (e.g., ethanol, butanol), hydrocarbons, and organic acids (e.g., acetic acid, lactic acid, butyric acid), for example. Such carbon-containing materials may be found in plant matter (e.g., corn, sugar beets, and bagasse), wastewater, manure, and compost, for example, which may be obtained from food and drink processing facilities (e.g., wineries) and farms, for example.

In the event that the carbon-containing material (e.g., inorganic carbon) cannot be consumed directly by the aquatic plants 106, suitable bacteria and/or fungus may be provided to convert the material to a form suitable for heterotrophic consumption by the aquatic plants 106 (e.g., organic carbon). Exemplary bacteria strains include, but are not limited to, Ralstonia eutropha and Pyrococcus furiosus, for example. As discussed above, substrate 108 may be inoculated with such bacteria.

According to an exemplary embodiment of the present disclosure, the carbon-containing materials may be obtained as waste from one or more sources, making the carbon-containing materials inexpensive and readily available. For example, the carbon-containing materials may be obtained as waste from food and drink processing facilities (e.g., wineries) and farms. The waste materials may be pretreated before introducing the waste materials to container 102 via the organic or inorganic carbon matter inlet 168. For example, liquid waste materials may be filtered, pressed, or otherwise processed to remove suspended solids. Solid waste materials may be separated to remove chunks to create fines for processing/use.

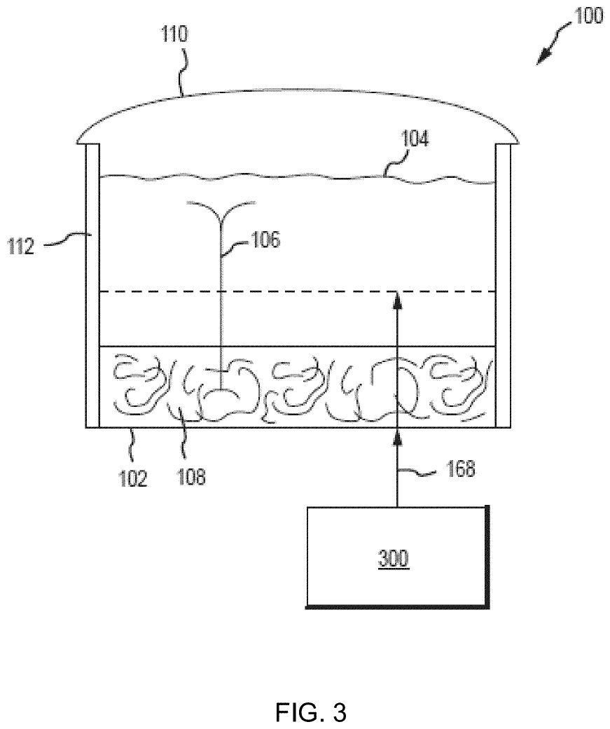

As illustrated in FIG. 3, the carbon-containing material may be a waste stream that is obtained from an adjacent ethanol facility 300, such as a first-generation ethanol facility. The waste stream may be an ethanol vapor waste stream that is obtained from trucks loading material at the ethanol facility 300. Rather than burning or releasing the ethanol vapor waste stream to the atmosphere, the ethanol vapor waste stream may be bubbled into water 104 of container 102 via the organic or inorganic carbon matter inlet 168. The waste stream may also include thin stillage and pressings from the ethanol facility 300, for example. By introducing the waste stream to container 102, the aquatic plants 106 in container 102 may take in and convert the waste stream to glucose, and then ultimately back to ethanol, which may be labeled and sold as an advanced biofuel.

### Substrate

As discussed above with reference to FIG. 1, substrate 108 is provided in container 102 to anchor and support root growth of the aquatic plants 106. Substrate 108 may be constructed of an inert material, such as plastic, that remains stable in water 104 over time. Substrate 108 may also be heavy enough to sink in water 104 and support the aquatic plants 106 both horizontally and vertically in container 102, even as water 104 flows through container 102. Substrate 108 may also have a high surface area to support root growth of the aquatic plants 106 and to support bacteria, fungus, and algae in container 102.

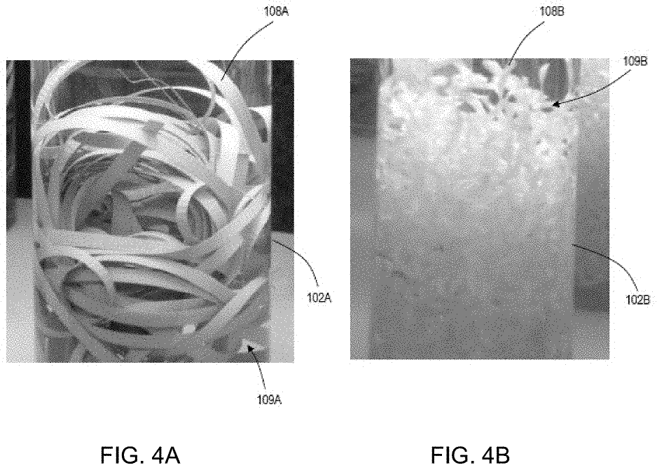

A first exemplary substrate 108A is shown in FIG. 4A. Substrate 108A can comprise one or more commercially-available elongate strips made of plastic material, such as packaging straps (e.g., box straps). The straps may be constructed of various plastic materials capable of forming elongate structures, such as polypropylene or other suitable material. In some cases, substrate 108A can be arranged so that it forms a wadded up mass. In other cases, the elongate plastic straps of substrate 108A can be arranged to form a net or mesh. Some materials may be made of a retardant material to reduce or prevent contamination by algae or other organisms. The dimensions of the straps may vary. For example, each strap may be about 0.25, 0.5, 1, or 2 inches in width or more. The straps are illustratively arranged irregularly (e.g., looped, twisted, and stacked) in the bottom of container 102A to provide open spaces or pores 109A between the straps to receive water, root growth, and other materials. Other configurations of substrate 108A are contemplated herein.

A second exemplary substrate 108B is shown in FIG. 4B. Substrate 108B comprises one or more commercially-available plastic ribbons, which may be formed by shaving a recycled plastic component. The ribbons are illustratively arranged in the bottom of container 102B to provide open spaces or pores 109B between the ribbons to receive water, root growth, and other materials.

In general, the substrate acts to anchor the root system of the plants and may comprise a region into which plant by-products such as ethanol are released. In one embodiment, the substrate includes a particulate material that serves as the primary anchoring mechanism. However, mechanical anchoring devices such as grids or screens, to which the roots may engage and couple themselves may be optionally used as well. The ratio of water depth to substrate thickness may range from about 2:1 to about 1:2. In one embodiment, the water depth may be less than or equal to the substrate thickness, for example in a water depth/substrate thickness ratio of about 1:1 or less, more particularly from about 1:1 to about 1:2. In a further embodiment, the water depth is less than the substrate thickness, for example in a water depth/substrate thickness ratio of less than 1:1.

In one embodiment, the substrate may use a coarse particulate formed from a porous mineral material. In other embodiments, the substrate may include two or more materials that may be formed as layers. The characteristics of each substrate layer can be configured as appropriate for the plant being used, including variations in chemical composition (e.g., nutrient content or pH), physical composition (e.g., coarseness or density), biological composition (e.g., bacteria), and the like.

Other exemplary substrates are described in U.S. Patent Application Publication No. 2013/0071902 to Hagen, filed Sep. 20, 2011, and U.S. Provisional Patent Application Ser. No. 61/943,943 to Hagen et al., filed Feb. 24, 2014, the disclosures of which are incorporated herein by reference in their entirety.

### Carbon Dioxide

As discussed above, carbon dioxide sources of use herein can include but are not limited to carbon dioxide from landfills, wastewater facilities, cement facilities, coal facilities (e.g. from syngas), naturally-occurring carbon dioxide or the like. These sources of carbon dioxide may be harvested from these facilities and delivered to a system or apparatus described herein.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A system for producing ethanol comprising:
a container;
a substrate in the container;
at least one water inlet to the container to deliver water to the container;
at least one carbon dioxide inlet to the container, the at least one carbon dioxide inlet communicating with an adjacent facility to receive carbon dioxide from the facility;
at least one aquatic plant having a root portion anchored to the substrate, the at least one aquatic plant performing a metabolic photosynthesis process to convert the carbon dioxide from the facility into glucose and a metabolic fermentation process to convert the glucose into ethanol; and
at least one outlet from the container to remove the water and the ethanol from the container.

2. The system of claim 1, further comprising a separator downstream of the facility and upstream of the container to dewater and cool the carbon dioxide from the facility.

3. The system of claim 1, wherein the at least one carbon dioxide inlet directs the carbon dioxide from the facility into a gaseous headspace above the water in the container.

4. The system of claim 1, wherein the at least one carbon dioxide inlet directs the carbon dioxide from the facility into the water in the container.

5. The system of claim 1, further comprising a reactor downstream of the facility and upstream of the container to convert the carbon dioxide from the facility into a bicarbonate material or a carbonate material.

6. The system of claim 1, wherein the metabolic photosynthesis process of the at least one aquatic plant produces oxygen in the container, the system further comprising an oxygen outlet from the container that communicates with the facility to direct oxygen from the container to the facility.

7. The system of claim 1, wherein the at least one aquatic plant comprises a submersed aquatic herb.

8. The system of claim 7, wherein the at least one aquatic plant comprises Potamogeton pectinatus.

9. The system of claim 1, wherein the at least one aquatic plant is genetically modified.

10. The system of claim 1, wherein the facility comprises a combustion facility, a landfill, wastewater, coal refinery, or cement facility.

11. A system for producing ethanol comprising:
a container;
a substrate in the container, the substrate comprising at least one elongate plastic material arranged irregularly in the container to define a plurality of spaces;
at least one water inlet to the container to deliver water to the container;
at least one aquatic plant anchored into the plurality of spaces in the substrate; and at least one outlet from the container to remove ethanol and the water from the container.

12. The system of claim 11 wherein the at least one elongate material is a packaging strap.

13. The system of claim 11, wherein the at least one elongate material is a ribbon shaved from a plastic component.

14. The system of claim 11, wherein the substrate is heavier than a such that the substrate sinks in the water in the container.

15. The system of claim 11, wherein the substrate is inoculated with at least one anaerobic bacteria.

16. The system of claim 11, further comprising at least one carbon dioxide inlet to the container and/or at least one carbon matter inlet to the container, wherein the at least one carbon dioxide inlet is in communication with an adjacent facility and configured to receive carbon dioxide from the facility, and wherein the at least one carbon matter inlet is in communication with an adjacent facility and configured to receive a waste carbon material from the adjacent facility.

17. The system of claim 16, wherein the waste carbon material is derived from plant matter, wastewater, manure, compost, or a combination thereof.

18. The system of claim 16, wherein the adjacent facility is an ethanol facility.

19. The system of claim 16, wherein the waste carbon material comprises ethanol vapor and/or liquid.

20. The system of claim 16, wherein he adjacent facility is a food processing facility, a wastewater facility, or a landfill.

21. The system of claim 16, wherein the at least one carbon matter inlet directs the waste carbon material into the water in the container.

22. The system of claim 16, wherein the at least one carbon matter inlet directs the waste carbon material into the substrate.

23. A system for producing ethanol comprising:
a container;
a substrate in the container, the substrate comprising at least one elongate plastic material arranged irregularly in the container to define a plurality of spaces;
at least one aquatic plant anchored into the plurality of spaces in the substrate,
at least one water inlet to the container to deliver water to the container;
at least one water outlet from the container to remove water and ethanol from the container.
